(19) United States
(12) Plant Patent Application Publication
(10) Pub. No.: US 2004/0268456 P1
(43) Pub. Date: Dec. 30, 2004

(54) Chrysanthemum plant named 'Chanizet'

(75) Inventor: Jean-Pierre Challet, Nuaille (FR)

Correspondence Address: BURNS DOANE SWECKER & MATHIS L L P, POST OFFICE BOX 1404, ALEXANDRIA, VA, 22313-1404, US

(73) Assignee: SELECTION NEW PLANT SARL, Le Luc (FR)

(21) Appl. No.: 10/893,910

(22) Filed: Jul. 20, 2004

Related U.S. Application Data
(63) Continuation of application No. 10/114,447, filed on Apr. 3, 2002.

(51) Int. Cl. A01H 5/00

(52) U.S. Cl. PLT/284

(57) ABSTRACT

A new and distinct Chrysanthemum cultivar is provided that is the result of a controlled breeding program. Attractive ball-shaped bronze blossoms are formed in profusion. Such blossoms commonly are approximately 10 to 12 cm in diameter when fully open. The growth habit is low and compact and well-branched and yields a generally uniform and spherical overall plant. The foliage is glossy dark green and contrasts well with the bronze blossom coloration, and the stems are extremely stiff. The plant grows well in pots and is well suited for use as an attractive potted gift plant.

This is a Continuation of U.S. Plant patent application Ser. No. 10/114,447, filed Apr. 3, 2002.

### BOTANICAL/COMMERCIAL CLASSIFICATION

Chrysanthemum mortifolium/Decorative Pot Mum

### VARIETAL DENOMINATION

cv. ‘Chanizet’

### SUMMARY OF THE INVENTION

The present invention comprises a new and distinct cultivar of Chrysanthemum, botanically known as Chrysanthemum mortifolium, and hereafter is referred to by the cultivar name ‘Chanizet’.

The new cultivar of the present invention was created at Nuaillé, France during the course of a controlled breeding program. The female parent was named ‘88/28/3’ (non-patented in the United States) and the male parent was named ‘87/172/4’ (non-patented in the United States). The seeds resulting from the cross were sown and small plants were obtained which were physically and biologically different from each other. Selective study resulted in the identification of a single plant of the new variety of the present invention.

It was found that the new Chrysanthemum cultivar of the present invention displays:

(a) a low compact, well-branched, and generally spherical growth habit,

(b) forms attractive long-lasting decorative ball-shaped bronze blossoms,

(c) forms extremely stiff stems,

(d) forms attractive dark green leaves having shiny upper surfaces, and

(e) an ability to grow well in pots to provide an attractive potted gift plant.

The new cultivar can be grown singly or in clumps in pots. It also can be grown in the landscape. The bronze blossoms contract nicely with the shiny dark green foliage. The blossoms have been found to be long lasting and commonly last approximately four weeks on the plant. Pinching is helpful to further enhance branching; however, such pinching is not necessary since the plant already is naturally self-branching.

The new ‘Chanizet’ cultivar of the present invention can be readily distinguished from its female and male parents. While each of the parental plants form bronze-incurred blossoms, the new cultivar displays stronger stems and darker green more healthy-appearing foliage.

The new ‘Chanizet’ cultivar can be readily distinguished from the ‘Chamidi’ cultivar (U.S. Plant patent Ser. No. 10/114,446, filed concurrently herewith) in view of the different blossom coloration. The blossoms of the ‘Chanizet’ cultivar of the present invention are a bronze while those of the ‘Chamidi’ parent cultivar are bright yellow in coloration. The ‘Chamidi’ cultivar is a mutation of the ‘Chanizet’ cultivar.

Asexual reproduction of the new cultivar by the use of cuttings as performed at Nuaillé, France, in a controlled environment has demonstrated that the characteristics of the new cultivar are firmly fixed and are retained through successive generations of asexual propagation.

‘Chanizet’ has not been observed under all possible environmental conditions to date. Accordingly, it is possible that the phenotype may vary somewhat with variations in the environment, such as temperature, light, day length, contact with pesticides and/or subjection to grown regulation treatments.

### BRIEF DESCRIPTION OF THE PHOTOGRAPHS

The accompanying photographs depict important parts of the new ‘Chanizet’ cultivar when grown in a pot in a greenhouse at Nuaillé, France. The plant was approximately four months of age.

FIG. 1 shows a close view of a typical attractive ball-shaped bronze blossom of the ‘Chanizet’ cultivar.

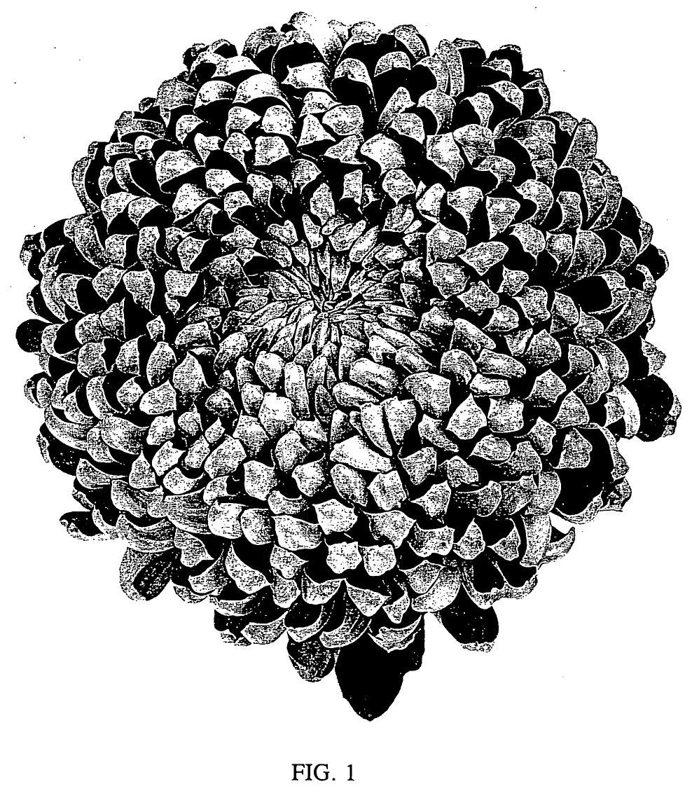

FIG. 2 shows a typical dark green leaf of the ‘Chanizet’ cultivar. While not readily apparent in a photograph of this character, the upper surface is glossy.

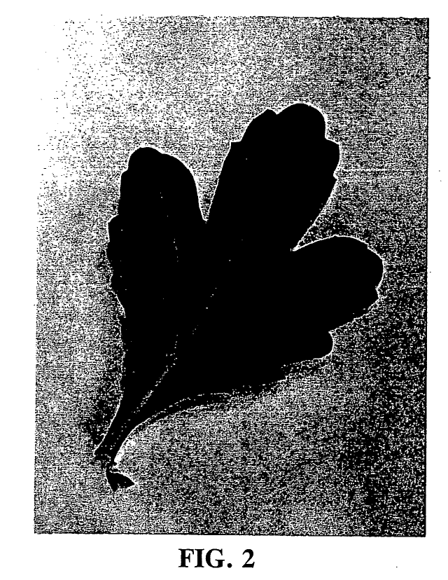

### DETAILED DESCRIPTION

The plants described were approximately four months of age and were grown in pots in a greenhouse at Nuaillé, France. No growth regulation was used. The growing conditions approximate those commonly utilized for the commercial production of decorative pot mums. Reference to the R.H.S. Colour Chart of The Royal Horticultural Society, London, England is included to designate color with specificity.

General appearance: Short and stocky with a generally spherical growth habit.

Foliage: Attractive dark green. The upper and under surfaces of mature leaves are near Green Group 137A. The upper surfaces of the leaves also are shiny and tend to glisten in the sunlight. The leaf texture is smooth and fleshy. A typical leaf commonly displays an average length of approximately 7 to 10 cm and an average width of approximately 4 to 6 cm. The leaf apex is very finely murconate and the leaf base is attenuate. The petiole is near Green Group 137A and 139A in coloration. The venation is slightly lighter green (as illustrated). The leaf margin bears medium serration (as illustrated). The petiole commonly is approximately 1.5 to 2.5 cm in length on average, and commonly is approximately 0.5 to 0.8 cm in diameter on average.

Stems: Extremely stiff and smooth in texture. A typical flowering stem commonly is approximately 35 to 40 cm in length on average, and approximately 1.1 to 1.4 cm in diameter on average. The internode length commonly is approximately 2.5 to 3.5 cm on average.

Flowers: There commonly are approximately 6 to 8 flowers per stem. They possess a decorative bronze coloration. There are no disc florets. The ray florets are concave in configuration and are near Greyed-Yellow Group 160B on the outside and near Red Group 42B with slightly more red on the inside. The ray floret apex commonly is pointed and occasionally is dentate. The ray florets commonly number at least 300 and are tubular at the base. The texture of the ray florets is somewhat roughened. The corolla tube also is near Greyed-Red Group 160B in coloration. The flower configuration is generally ball-shaped and the diameter is approximately 10 to 12 cm on average when fully expanded. A typical ray floret commonly displays an average length of approximately 7 to 13 cm and an average width of approximately 1 to 1.4 cm. The corolla tube commonly is approximately 6 to 11.5 cm in length on average. The buds are rounded at the tip, approximately 1.6 to 2.2 cm in length on average, approximately 2.5 to 3.5 cm in diameter on average, and near Greyed-Purple Group 183C and 184B in coloration. The peduncle is very strong, possesses a smooth texture, possesses a length of approximately 5 to 7 cm on average, possesses a diameter of approximately 0.8 to 1.2 cm on average, and possesses a coloration of near Yellow-Green Group 146B. No reproductive organs or seeds have been observed to date.

Flower duration: Very long lasting with the blossoms commonly lasting approximately four weeks on the plant.

Response time: The time between the beginning of the short day period and the flowering date at normal temperature and light intensity is approximately ten weeks.

Hardiness: Has satisfactorily withstood temperatures in the range of 0 to 2° C.

Usage: Decorative pot mum. No noteworthy resistance or susceptibility to disease, insects, or drought has been encountered during observations to date.

I claim:

1. A new and distinct cultivar of Chrysanthemum plant that displays:
(a) a low compact, well-branched, and generally spherical growth habit, p1 (b) forms attractive long-lasting decorative ball-shaped bronze blossoms,
(c) forms extremely stiff stems,
(d) forms attractive dark green leaves having shiny upper surfaces, and
(e) an ability to grow well in pots to provide an attractive potted gift plant;
substantially as illustrated and described.
